(19) United States
(12) Patent Application Publication
(10) Pub. No.: US 2023/0008508 A1
(43) Pub. Date: Jan. 12, 2023

(54) COMBINATION THERAPIES FOR THE TREATMENT OF CANCER

(71) Applicant: H. LEE MOFFITT CANCER CENTER AND RESEARCH INSTITUTE, INC., Tampa, FL (US)

(72) Inventors: Elsa FLORES, Tampa, FL (US); Ken TSAI, Tampa, FL (US)

(21) Appl. No.: 17/783,937

(22) PCT Filed: Dec. 10, 2020
(86) PCT No.: PCT/US2020/064291

§ 371 Date: Jun. 9, 2022

Related U.S. Application Data
(60) Provisional application No. 62/946,036, filed on Dec. 10, 2019.

(51) Int. Cl. A61K 31/713; A61K 31/4045; A61P 35/00

(52) U.S. Cl. CPC A61K 31/713; A61K 31/4045; A61P 35/00

(57) ABSTRACT

Disclosed are compositions comprising a glycolysis inhibitor and a HDAC inhibitor and methods for treatment of cancer using said compositions.

### I. BACKGROUND

TP53 is commonly altered in human cancer, and Tp53 reactivation suppresses tumors in-vivo. Thus, targeting this tumor suppressor pathway is highly desirable and will impact many cancer patients. However, this strategy has proven difficult to implement therapeutically. The TA isoforms of p63 and p73 structurally and functionally resemble p53, while the ΔN isoforms of p63 and p73 are frequently overexpressed in cancer and act primarily in dominant negative fashion against p53, TAp63, and TAp73 to inhibit their tumor suppressive functions. The p53 family interacts extensively in cellular processes that promote tumor suppression, such as apoptosis and autophagy, thus a clear understanding of this interplay in cancer is needed to treat tumors with alterations in the p53 pathway.

### II. SUMMARY

Disclosed are methods and compositions related to combination therapies that target the p53 family members and methods of treating cancer utilizing said combination therapies.

In one aspect disclosed herein are combination therapies comprising a glycolysis inhibitor (such as, for example, an amylin analog including but not limited to pramlintide) and an histone deacetylase (HDAC) inhibitor (HDACi) (such as, for example, hydroxamic acids including, but not limited to panobinostat, belinostat, trichostatin A, givinostat, resminostat, abexinostat, quisinostat, rocilinostat, practinostat, CHR-3996 and/or vorinostat; short chain fatty acids including, but not limited to valproic acid, butyric acid, and/or phenylbutyric acid; benzamides including, but not limited to entinostat, tacedinaline, 4SC202, and/or mocetinostat; cyclic tetrapeptides including, but not limited to romidepsin; and/or sirtuins inhibitors including, but not limited to nicotinamide, sirtinol, cambinol, and/or E-527).

Also disclosed herein are pharmaceutical compositions comprising a glycolysis inhibitor (such as, for example, an amylin analog including but not limited to pramlintide) and an histone deacetylase (HDAC) inhibitor (HDACi) (such as, for example, hydroxamic acids including, but not limited to panobinostat, belinostat, trichostatin A, givinostat, resminostat, abexinostat, quisinostat, rocilinostat, practinostat, CHR-3996 and/or vorinostat; short chain fatty acids including, but not limited to valproic acid, butyric acid, and/or phenylbutyric acid; benzamides including, but not limited to entinostat, tacedinaline, 4SC202, and/or mocetinostat; cyclic tetrapeptides including, but not limited to romidepsin; and/or sirtuins inhibitors including, but not limited to nicotinamide, sirtinol, cambinol, and/or E-527).

In one aspect, disclosed herein are methods of treating, inhibiting, reducing, and/or preventing a cancer or metastasis (such as for example, cutaneous squamous cell carcinoma (cuSCC), head and neck squamous cell carcinoma (HNSCC), or lung cancer or any cancer comprising a p53, p63, or p73 mutation or aberrant expression thereof) in a subject comprising administering to the subject the combination therapy or the pharmaceutical composition any preceding aspect. For example, disclosed herein are methods of treating, inhibiting, reducing, and/or preventing a cancer or metastasis (such as for example, cutaneous squamous cell carcinoma (cuSCC, HNSCC, or lung cancer) in a subject comprising administering to the subject a first and second agent that inhibit ΔNp63 and/or ΔNp73; wherein the first agent comprises a glycolysis inhibitor (such as, for example, an amylin analog including but not limited to pramlintide); and wherein the second agent comprises an histone deacetylase (HDAC) inhibitor (HDACi) (such as, for example, hydroxamic acids including, but not limited to panobinostat, belinostat, trichostatin A, givinostat, resminostat, abexinostat, quisinostat, rocilinostat, practinostat, CHR-3996 and/or vorinostat; short chain fatty acids including, but not limited to valproic acid, butyric acid, and/or phenylbutyric acid; benzamides including, but not limited to entinostat, tacedinaline, 4SC202, and/or mocetinostat; cyclic tetrapeptides including, but not limited to romidepsin; and/or sirtuins inhibitors including, but not limited to nicotinamide, sirtinol, cambinol, and/or E-527). In one aspect, disclosed herein are methods of treating a cancer of any preceding aspect, wherein the method further comprises administering to the subject a radiation therapy.

6. Also disclosed herein are cancer treatment, inhibition, reduction, and/or prevention regimens comprising a) detecting the expression of Fxbw7 in the cancer (such as for example, cutaneous squamous cell carcinoma (cuSCC), head and neck squamous cell carcinoma (HNSCC), or lung cancer or any cancer comprising a p53, p63, or p73 mutation or aberrant expression thereof); wherein expression of Fxbw7 indicates susceptibility of the cancer to inhibitors of ΔNp63 and/or ΔNp73; and b) when the presence of Fxbw7 expression is detected, the method further comprises administering to the subject a glycolysis inhibitor (such as, for example, an amylin analog including but not limited to pramlintide) and/or a histone deacetylase (HDAC) inhibitor (HDACi) (such as, for example, hydroxamic acids including, but not limited to panobinostat, belinostat, trichostatin A, givinostat, resminostat, abexinostat, quisinostat, rocilinostat, practinostat, CHR-3996 and/or vorinostat; short chain fatty acids including, but not limited to valproic acid, butyric acid, and/or phenylbutyric acid; benzamides including, but not limited to entinostat, tacedinaline, 4SC202, and/or mocetinostat; cyclic tetrapeptides including, but not limited to romidepsin; and/or sirtuins inhibitors including, but not limited to nicotinamide, sirtinol, cambinol, and/or E-527); or c) when the presence of Fxbw7 expression is not detected, the method further comprises administering to the subject a cancer regimen that does not include an agent that does not inhibit inhibitors of ΔNp63 and/or ΔNp73.

In one aspect, disclosed herein are methods of increasing the sensitivity of a cancer (such as for example, cutaneous squamous cell carcinoma (cuSCC), head and neck squamous cell carcinoma (HNSCC), or lung cancer or any cancer comprising a p53, p63, or p73 mutation or aberrant expression thereof) in a subject to histone deacetylase (HDAC) inhibitor (HDACi) therapy (such as, for example, hydroxamic acids including, but not limited to panobinostat, belinostat, trichostatin A, givinostat, resminostat, abexinostat, quisinostat, rocilinostat, practinostat, CHR-3996 and/or vorinostat; short chain fatty acids including, but not limited to valproic acid, butyric acid, and/or phenylbutyric acid; benzamides including, but not limited to entinostat, tacedinaline, 4SC202, and/or mocetinostat; cyclic tetrapeptides including, but not limited to romidepsin; and/or sirtuins inhibitors including, but not limited to nicotinamide, sirtinol, cambinol, and/or E-527) comprising administering to the subject an agent that inhibits glycolysis (such as, for example, an amylin analog including but not limited to pramlintide). It is understood and herein contemplated that the administration of the glycolysis inhibitor decreases the effective concentration needed for the HDACi to be effective against the cancer. Thus, in one aspect, disclosed herein are methods of increasing the sensitivity of a cancer to HDACi therapy of any preceding aspect, wherein administration of the glycolysis inhibitor decrease the inhibitory concentration of the HDACi needed to be effective against the cancer

A method of increasing the sensitivity of a cancer (such as for example, cutaneous squamous cell carcinoma (cuSCC), head and neck squamous cell carcinoma (HNSCC), or lung cancer or any cancer comprising a p53, p63, or p73 mutation or aberrant expression thereof) in a subject to radiation therapy comprising administering to the subject an agent that inhibits ΔNp63 and/or ΔNp73. In one aspect the agent that inhibits ΔNp63 and/or ΔNp73 comprises an inhibitor of glycolysis (such as, for example, an amylin analog including but not limited to pramlintide).

Also disclosed herein are methods of detecting sensitivity of a cancer (such as for example, cutaneous squamous cell carcinoma (cuSCC), head and neck squamous cell carcinoma (HNSCC), or lung cancer or any cancer comprising a p53, p63, or p73 mutation or aberrant expression thereof) to a treatment that suppresses of ΔNp63 and/or ΔNp73 comprising detecting the presence of Fbxw7 expression; wherein the presence of Fbxw7 expression indicates sensitivity to a treatment that suppresses ΔNp63 and/or ΔNp73. In one aspect, the treatment that suppresses ΔNp63 and/or ΔNp73 comprises a glycolysis inhibitor (such as, for example, an amylin analog including but not limited to pramlintide) and/or a histone deacetylase (HDAC) inhibitor (HDACi) such as, for example, hydroxamic acids including, but not limited to panobinostat, belinostat, trichostatin A, givinostat, resminostat, abexinostat, quisinostat, rocilinostat, practinostat, CHR-3996 and/or vorinostat; short chain fatty acids including, but not limited to valproic acid, butyric acid, and/or phenylbutyric acid; benzamides including, but not limited to entinostat, tacedinaline, 4SC202, and/or mocetinostat; cyclic tetrapeptides including, but not limited to romidepsin; and/or sirtuins inhibitors including, but not limited to nicotinamide, sirtinol, cambinol, and/or E-527).

In one aspect, disclosed herein are methods of detecting sensitivity of a cancer (such as for example, cuSCC, HNSCC, or lung cancer) to a treatment that suppresses of ΔNp63 and/or ΔNp73 (such as, for example, an amylin analog including but not limited to pramlintide) and/or a histone deacetylase (HDAC) inhibitor (HDACi) such as, for example, hydroxamic acids including, but not limited to panobinostat, belinostat, trichostatin A, givinostat, resminostat, abexinostat, quisinostat, rocilinostat, practinostat, CHR-3996 and/or vorinostat; short chain fatty acids including, but not limited to valproic acid, butyric acid, and/or phenylbutyric acid; benzamides including, but not limited to entinostat, tacedinaline, 4SC202, and/or mocetinostat; cyclic tetrapeptides including, but not limited to romidepsin; and/or sirtuins inhibitors including, but not limited to nicotinamide, sirtinol, cambinol, and/or E-527) comprising obtaining a tissue sample, detecting the transcriptional level of connective tissue growth factor (CTGF), Cysteine-rich angiogenic inducer 61 (CYR61), or F-box protein 16 (FBXO16) in the tissue sample, wherein a decrease of CTFG or CYR61 or an increase of FBXO16 relative to a normal control indicates that the cancerous tissue is sensitive to a treatment that suppresses of ΔNp63; and wherein an increase of CTFG or CYR61 or a decrease of FBXO16 relative to a normal control indicates that the cancerous tissue is resistant to a treatment that suppresses of ΔNp63.

Also disclosed herein are methods of detecting sensitivity of a cancer (such as for example, cuSCC, HNSCC, or lung cancer) to a treatment that suppresses of ΔNp63 and/or ΔNp73 (such as, for example, an amylin analog including but not limited to pramlintide) and/or a histone deacetylase (HDAC) inhibitor (HDACi) such as, for example, hydroxamic acids including, but not limited to panobinostat, belinostat, trichostatin A, givinostat, resminostat, abexinostat, quisinostat, rocilinostat, practinostat, CHR-3996 and/or vorinostat; short chain fatty acids including, but not limited to valproic acid, butyric acid, and/or phenylbutyric acid; benzamides including, but not limited to entinostat, tacedinaline, 4SC202, and/or mocetinostat; cyclic tetrapeptides including, but not limited to romidepsin; and/or sirtuins inhibitors including, but not limited to nicotinamide, sirtinol, cambinol, and/or E-527) comprising obtaining a tissue sample, contacting the tissue sample with a glycolysis inhibitor (such as, for example pramlintide); measuring the level of connective tissue growth factor (CTGF), Cysteine-rich angiogenic inducer 61 (CYR61), or F-box protein 16 (FBXO16) in the tissue sample, wherein a decrease of CTFG or CYR61 or an increase of FBXO16 relative to a normal control indicates that the cancerous tissue is sensitive to a treatment that suppresses of ΔNp63; and wherein no change or an increase of CTFG or CYR61 or a decrease of FBXO16 relative to a normal control indicates that the cancerous tissue is resistant to a treatment that suppresses of ΔNp63.

### III. BRIEF DESCRIPTION OF THE DRAWINGS

The accompanying drawings, which are incorporated in and constitute a part of this specification, illustrate several embodiments and together with the description illustrate the disclosed compositions and methods.

FIG. 1 shows an RNA sequence array showing the genes involved in metabolism upon inactivation of ΔNp63 or ΔNp73.

FIGS. 2A, 2B, 2C, and 2D show that the sensitivity to pramlintide is related to suppression of glycolysis. FIG. 2A shows an inhibition curve for several cutaneous SCC cell lines and shows inhibition of glycolysis for sensitive lines vs. resistant ones which do not exhibit this decrease in glycolysis. FIG. 2B shows the effect of pramlintide on the glycolysis of sensitive cutaneous squamous cell carcinoma (cuSCC) cell lines. Glycolysis, as measured by Extracellular Acidification Rate (Seahorse), is diminished by pramlintide administration (10 micromolar) in the sensitive SQ20B and FaDu Head and Neck SCC lines. FIG. 2C shows the effect of pramlintide on the glycolysis of resistant cutaneous squamous cell carcinoma (cuSCC) cell lines. FIG. 2D shows the effect of pramlintide on the glycolysis of sensitive head and neck squamous cell carcinoma (HNSCC) cell lines. Glycolysis, as measured by Extracellular Acidification Rate (Seahorse), is diminished by pramlintide administration (10 micromolar) in the sensitive SQ20B and FaDu Head and Neck SCC lines.

15. FIG. 3 shows that Pramlintide induces tumor regression in spontaneous UV-driven cutaneous squamous cell carcinoma (cuSCC) model.

FIG. 4 shows that pramlintide suppresses ΔNp63 expression.

FIG. 5 shows that ΔNp63 targets CYR61 and CTGF were downregulated in sensitive cells and upregulated in resistant cells. Based on RNAseq data, we identified ΔNp63 transcriptional targets downregulated in sensitive cells and upregulated in resistant cells and identified CYR61 and CTGF.

FIG. 6 shows the validation of CYR61 and CTGF in cuSSC cell lines. Sensitive lines downregulate CYR61 and CTGF in response to both pramlintide and direct siRNA-mediated knockdown of ΔNp63 showing that pramlintide mediated degradation of ΔNp63 affects target gene expression. Resistant lines upregulate CYR61 and CTGF in response to both pramlintide and direct siRNA-mediate knockdown of ΔNp63.

FIG. 7 shows that the ΔNp63 target FBXO16 was upregulated in sensitive cells and downregulated in resistant cells. Based on RNAseq data, we identified ΔNp63 transcriptional targets upregulated in sensitive cells and downregulated in resistant cells and identified FBXO16.

FIG. 8 shows the validation of the FBXO16 findings in cuSCC cell lines. Sensitive lines upregulate FBXO16 in response to both pramlintide and direct siRNA-mediate knockdown of ΔNp63 showing that pramlintide mediated degradation of ΔNp63 affects target gene expression. Thus target genes may be used as a biomarker of responsiveness to pramlintide. Resistant lines downregulate FBXO16 in response to both pramlintide and direct siRNA-mediate knockdown of ΔNp63 showing that pramlintide mediated degradation of ΔNp63 affects target gene expression.

FIG. 9 shows the expression of ΔNp63 in other cancer cell types.

FIG. 10 shows the validation of downstream targets CYR61, CTGF, and FBXO16 in HNSCC cell lines.

FIG. 11 shows the effect of pramlintide (symlin) on ΔNp63 protein levels in the sensitive HNSCC cell line SQ20B.

FIG. 12 shows the effect of pramlintide (symlin) on ΔNp63protein levels in the sensitive HNSCC cell line FaDu.

FIG. 13 shows that Fbxw7 expression is important for the effect of pramlintide (Symlin) on ΔNp63 protein levels.

FIG. 14 shows pramlintide treatment reduces ΔNp63 protein levels in the lung squamous cell carcinoma (LUSC) HCC95 cells (lung cancer cell line).

FIG. 15 shows pramlintide and HDACi treatments reduce ΔNp63 in H1975 lung adenocarcinoma (LUAD) cells. Protein levels of ΔNp63 are diminished by pramlintide and HDACi in H1975 lung adenocarcinoma cell lines.

FIG. 16 shows the effect of pramlintide (symlin) and HDAC inhibitors panobinostat and romidepsin on cell proliferation and survival of H1975 cells. Cell cycle arrest and apoptosis are increased by pramlintide and HDACi in HCC95 lung SCC and H1975 lung adenocarcinoma cell lines.

FIG. 17 shows the effects of pramlintide (symlin) on the glycolytic rate of HCC95 and H1975 cells. Glycolysis as measured by ECAR in HCC95 lung SCC and H1975 lung adenocarcinoma cells are decreased by pramlintide.

FIG. 18 shows inhibitor curves demonstrating that the combination of HDACi and pramlintide improves responses to compared to either treatment alone. IC50 curves for pramlintide in the absence of panobinostat (orange) at the IC20 for panobinostat alone (yellow) and at the IC40 for panobinostat alone (blue).

FIG. 19 shows that the combination of HDACi and pramlintide cooperatively suppresses ΔNp63 expression.

FIG. 20 shows that Pramlintide induces ROS in prior published studies in-vivo in tumors.

FIG. 21 shows the effect of radiation treatment of SRB12 cells with and without pramlintide at the IC40.

### IV. DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

A. Definitions

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

“Optional” or “optionally” means that the subsequently described event or circumstance may or may not occur, and that the description includes instances where said event or circumstance occurs and instances where it does not.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

“Treat,” “treating,” “treatment,” and grammatical variations thereof as used herein, include the administration of a composition with the intent or purpose of partially or completely preventing, delaying, curing, healing, alleviating, relieving, altering, remedying, ameliorating, improving, stabilizing, mitigating, and/or reducing the intensity or frequency of one or more a diseases or conditions, a symptom of a disease or condition, or an underlying cause of a disease or condition. Treatments according to the invention may be applied preventively, prophylactically, pallatively or remedially. Prophylactic treatments are administered to a subject prior to onset (e.g., before obvious signs of cancer), during early onset (e.g., upon initial signs and symptoms of cancer), or after an established development of cancer. Prophylactic administration can occur for day(s) to years prior to the manifestation of symptoms of an infection.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

“Biocompatible” generally refers to a material and any metabolites or degradation products thereof that are generally non-toxic to the recipient and do not cause significant adverse effects to the subject.

“Comprising” is intended to mean that the compositions, methods, etc. include the recited elements, but do not exclude others. “Consisting essentially of” when used to define compositions and methods, shall mean including the recited elements, but excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions provided and/or claimed in this disclosure. Embodiments defined by each of these transition terms are within the scope of this disclosure.

A “control” is an alternative subject or sample used in an experiment for comparison purposes. A control can be “positive” or “negative.”

The term “subject” refers to any individual who is the target of administration or treatment. The subject can be a vertebrate, for example, a mammal. In one aspect, the subject can be human, non-human primate, bovine, equine, porcine, canine, or feline. The subject can also be a guinea pig, rat, hamster, rabbit, mouse, or mole. Thus, the subject can be a human or veterinary patient. The term “patient” refers to a subject under the treatment of a clinician, e.g., physician.

“Effective amount” of an agent refers to a sufficient amount of an agent to provide a desired effect. The amount of agent that is “effective” will vary from subject to subject, depending on many factors such as the age and general condition of the subject, the particular agent or agents, and the like. Thus, it is not always possible to specify a quantified “effective amount.” However, an appropriate “effective amount” in any subject case may be determined by one of ordinary skill in the art using routine experimentation. Also, as used herein, and unless specifically stated otherwise, an “effective amount” of an agent can also refer to an amount covering both therapeutically effective amounts and prophylactically effective amounts. An “effective amount” of an agent necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

A “pharmaceutically acceptable” component can refer to a component that is not biologically or otherwise undesirable, i.e., the component may be incorporated into a pharmaceutical formulation provided by the disclosure and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

“Pharmacologically active” (or simply “active”), as in a “pharmacologically active” derivative or analog, can refer to a derivative or analog (e.g., a salt, ester, amide, conjugate, metabolite, isomer, fragment, etc.) having the same type of pharmacological activity as the parent compound and approximately equivalent in degree.

“Therapeutic agent” refers to any composition that has a beneficial biological effect. Beneficial biological effects include both therapeutic effects, e.g., treatment of a disorder or other undesirable physiological condition, and prophylactic effects, e.g., prevention of a disorder or other undesirable physiological condition (e.g., a non-immunogenic cancer). The terms also encompass pharmaceutically acceptable, pharmacologically active derivatives of beneficial agents specifically mentioned herein, including, but not limited to, salts, esters, amides, proagents, active metabolites, isomers, fragments, analogs, and the like. When the terms “therapeutic agent” is used, then, or when a particular agent is specifically identified, it is to be understood that the term includes the agent per se as well as pharmaceutically acceptable, pharmacologically active salts, esters, amides, proagents, conjugates, active metabolites, isomers, fragments, analogs, etc.

“Therapeutically effective amount” or “therapeutically effective dose” of a composition (e.g. a composition comprising an agent) refers to an amount that is effective to achieve a desired therapeutic result. In some embodiments, a desired therapeutic result is the control of type I diabetes. In some embodiments, a desired therapeutic result is the control of obesity. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect, such as pain relief. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

B. Method of Treating Cancer

TP53 is commonly altered in human cancer, and TP53 reactivation suppresses tumors in-vivo. This strategy has proven difficult to implement therapeutically, and herein is examined an alternative strategy by manipulating the p53 family members, p63 and p73. The TA isoforms of p63 and p73 structurally and functionally resemble p53, while the AN isoforms of p63 and p73 are frequently overexpressed in cancer and act primarily in dominant negative fashion against p53, TAp63, and TAp73 to inhibit their tumor suppressive functions. The p53 family interacts extensively in cellular processes that promote tumor suppression, such as apoptosis and autophagy, thus a clear understanding of this interplay in cancer is needed to treat tumors with alterations in the p53 pathway.

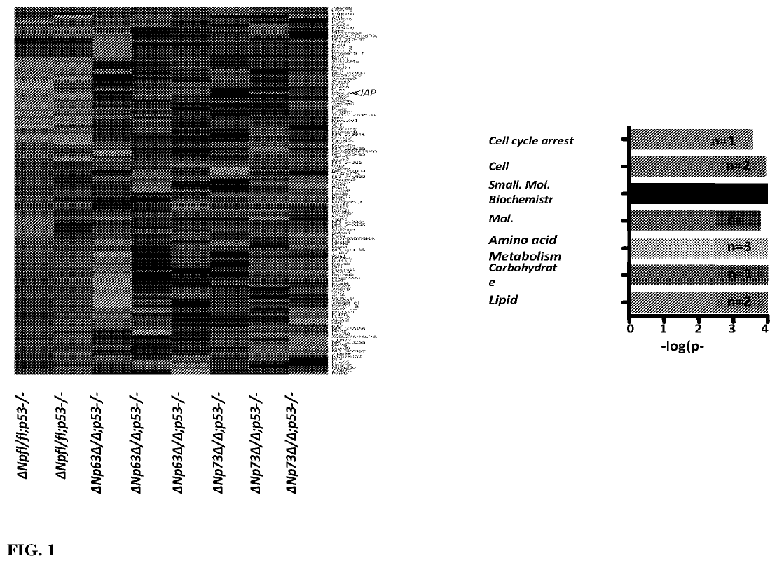

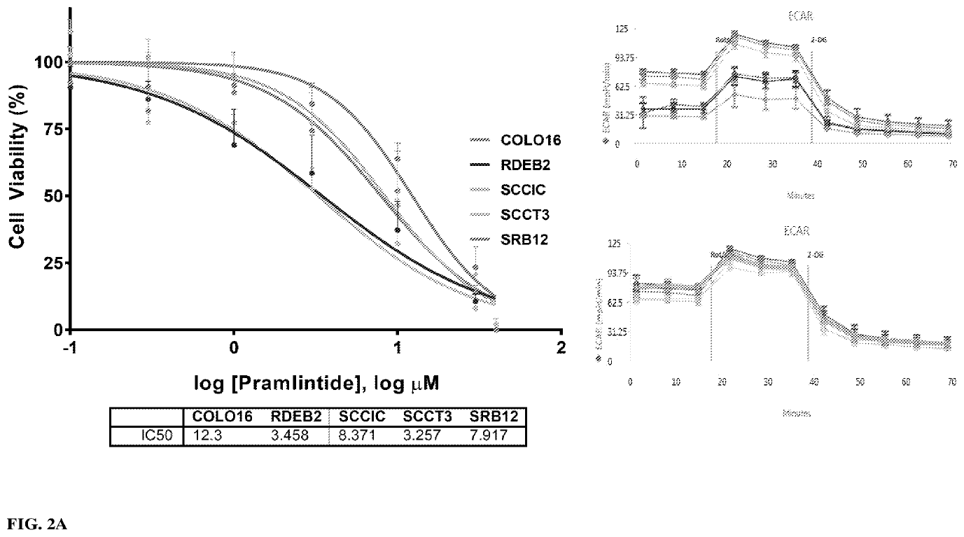

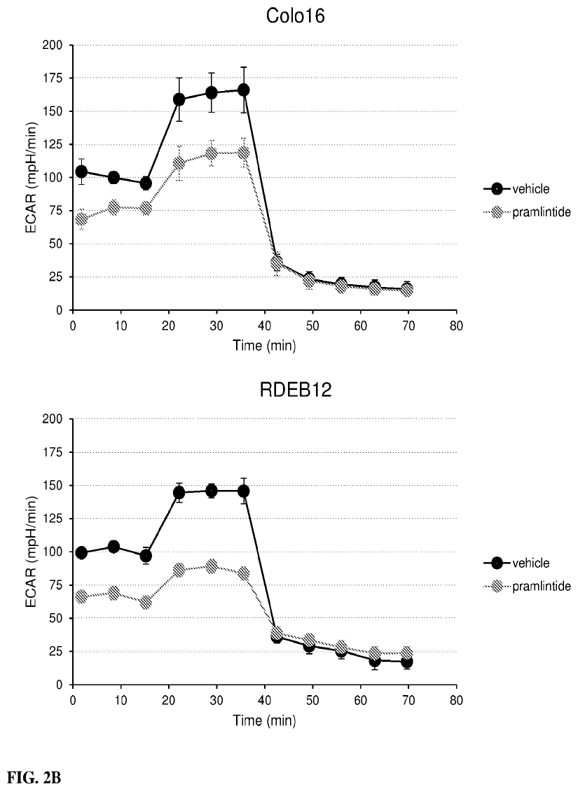

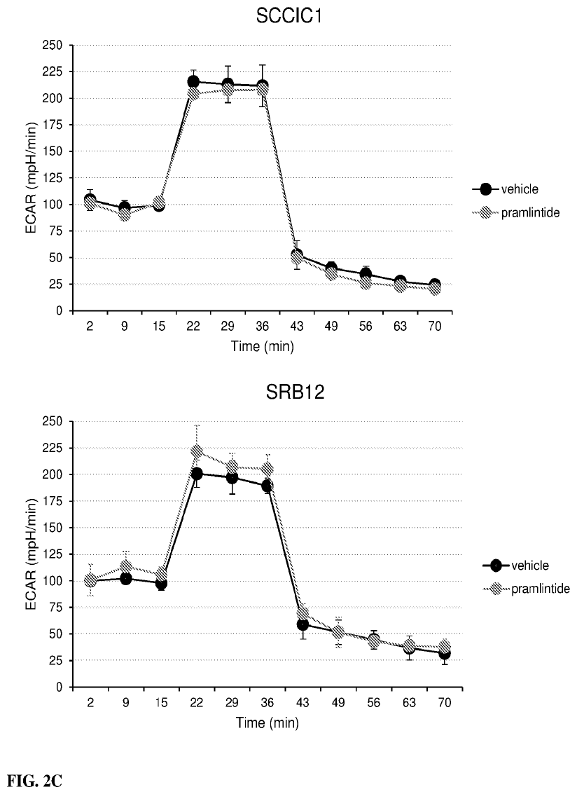

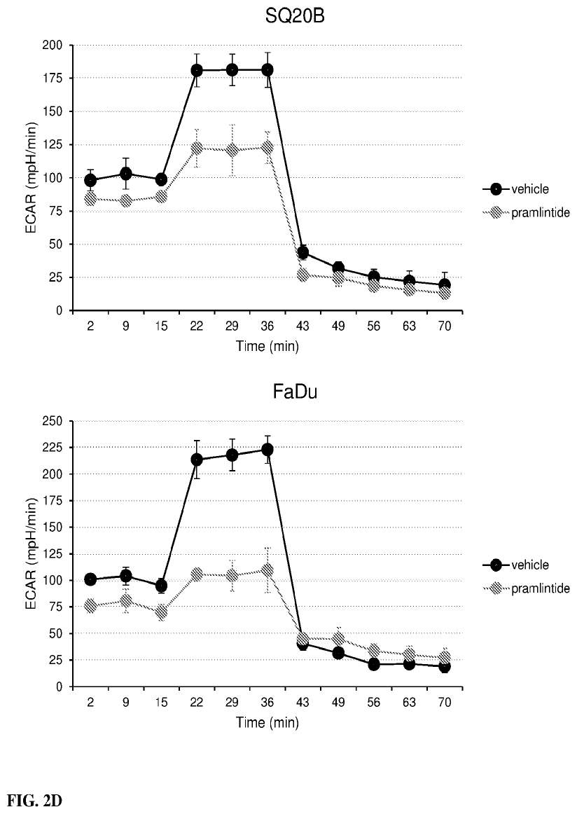

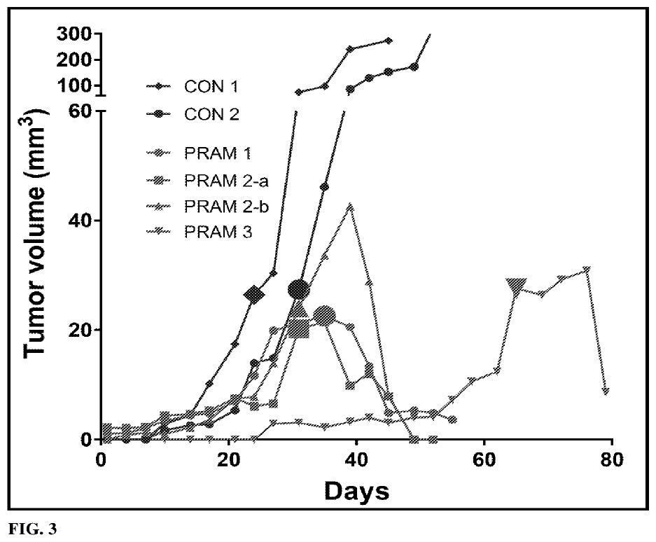

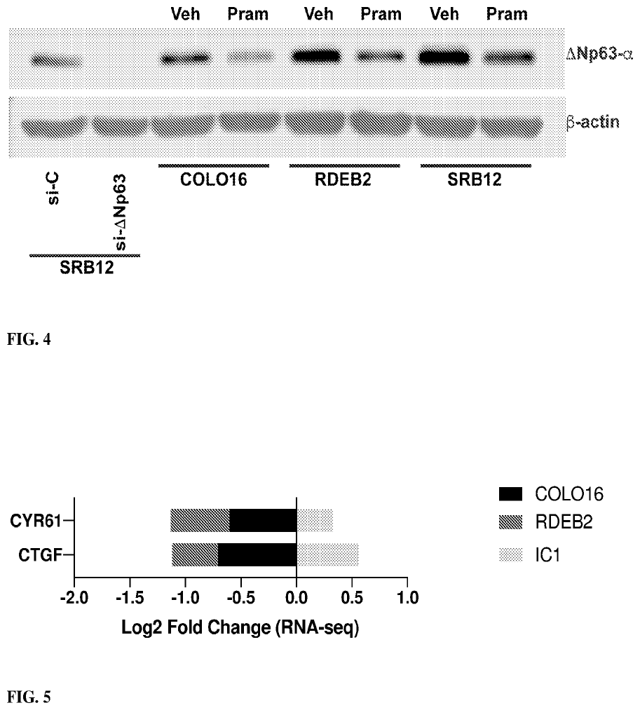

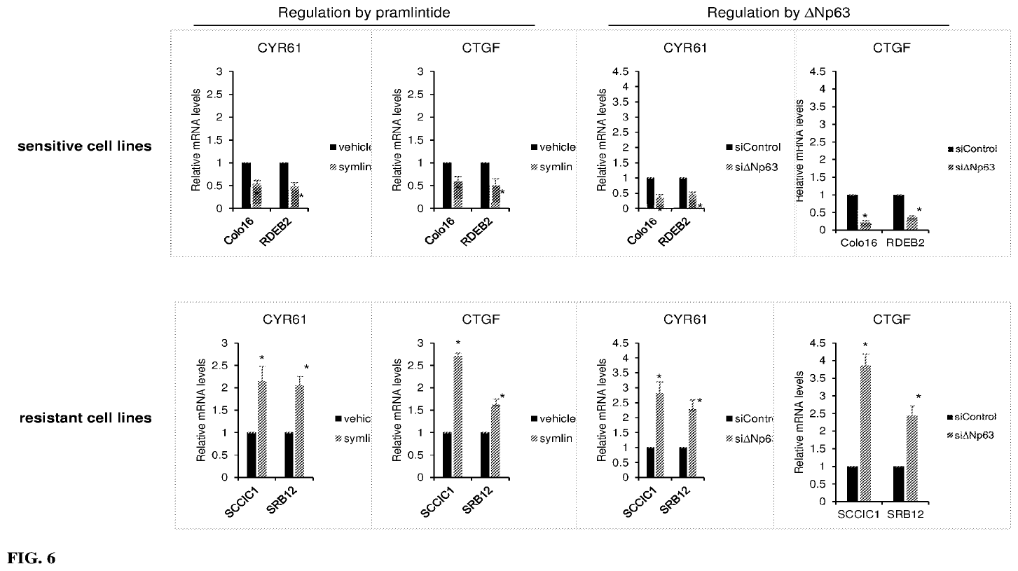

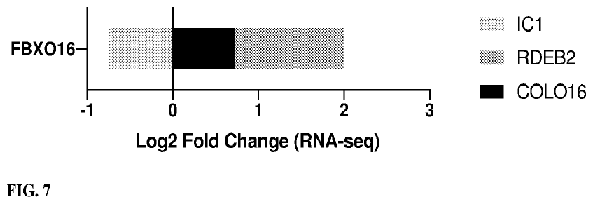

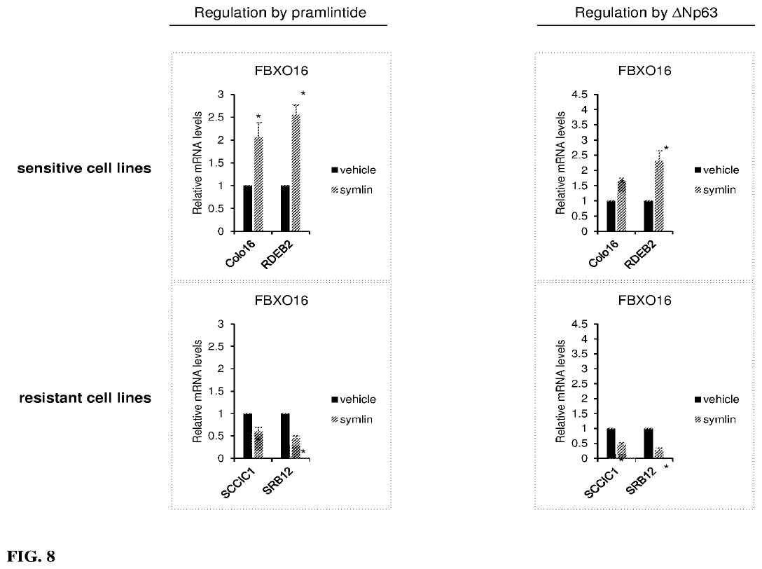

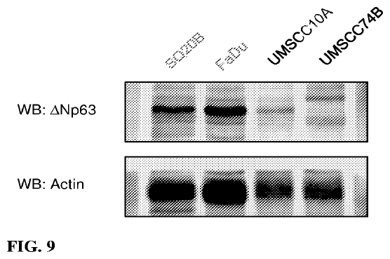

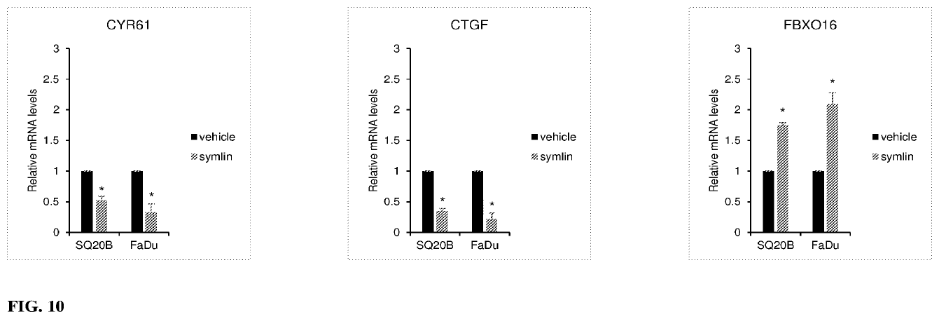

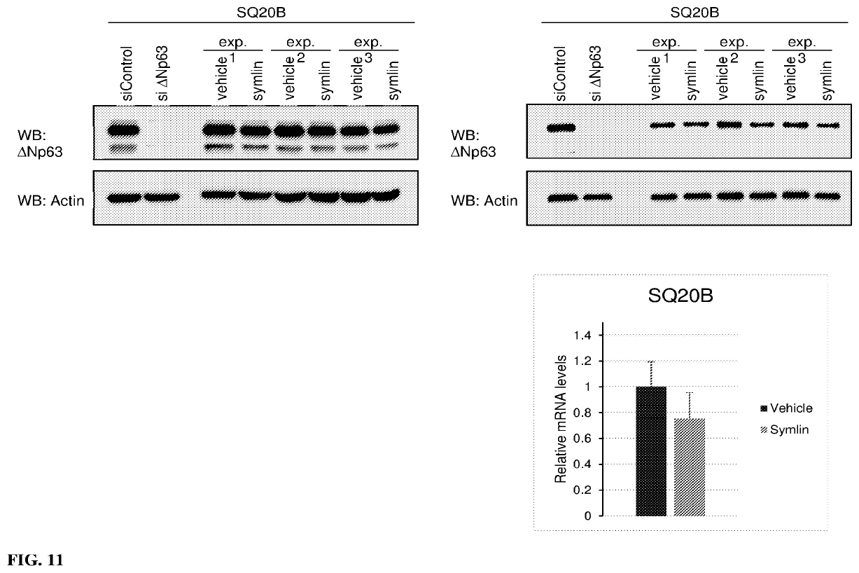

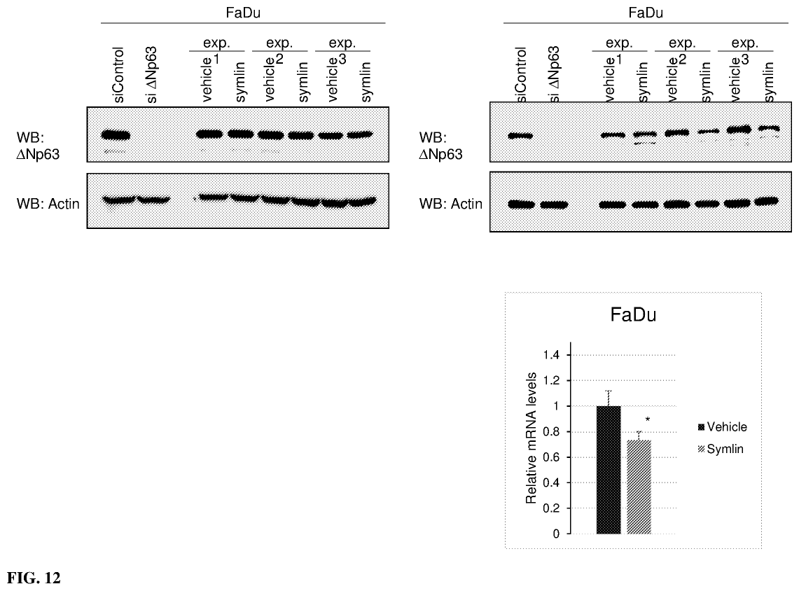

Here, is shown that deletion of the AN isoforms of p63 or p73 leads to metabolic reprogramming and regression of p53 deficient tumors (FIG. 1). Because ΔNp63 and ΔNp73 had an effect on glycolysis, Pramlintide, a synthetic analog of amylin, which is currently used to treat type 1 and type 2 diabetes was investigated for its effect on cancer. As shown in FIGS. 2A, 2B, 2C, and 2D, pramlintide suppresses glycolysis in sensitive cells. Additionally, pramlintide caused rapid tumor regression in p53 deficient thymic lymphomas, lung cancers, and cutaneous squamous cell carcinomas (FIG. 3), representing a novel strategy to target p53-deficient cancers. When investigated further it was shown that pramlintide suppresses 4Np63 expression (FIG. 4). Looking downstream of 4Np63 we observed that 4Np63 targets CYR61 and CTGF which were down regulated in sensitive cells and upregulated in resistant cells (FIG. 5). These findings were validated across the sensitive cell lines Colo16 and RDEB2 and the resistant cell lines SCCIC1 and SRB12 for pramlintide (symlin) administration and regulation by ΔNp63 showing reduced levels for CYR61 and CTGF in the sensitive lines and no effect or increases in resistant lines (FIG. 6). A similar observation was also made with the F-box protein FBXO16. There, FBXO16 was upregulated in sensitive cells and down regulated in resistant cells (FIGS. 7 and 8). Looking at additional cell types, ΔNp63 was expressed in sensitive cells but not resistant cells (FIG. 9). To see if the regulation pattern for CYR61, CTGF, and FBXO16 was maintained in HNSCC cell lines, the expression level for each was measured in SQ20B and FaDu cells with and without the application of pramlintide (symlin) (FIG. 10). As for the Sensitive cuSCC lines, the sensitive HNSCC lines upregulate FBXO16 and downregulate CYR61 and CTGF in response to both pramlintide showing that pramlintide mediated degradation of ΔNp63 affects target gene expression. Thus, target genes cay be used as a biomarker of responsiveness to pramlintide. We next investigated the effect of pramlintide (symlin) on ΔNp63 expression levels in SQ20B (FIG. 11) and FaDu (FIG. 12) cell lines. We observed decreased protein expression in western blots for both cell lines.

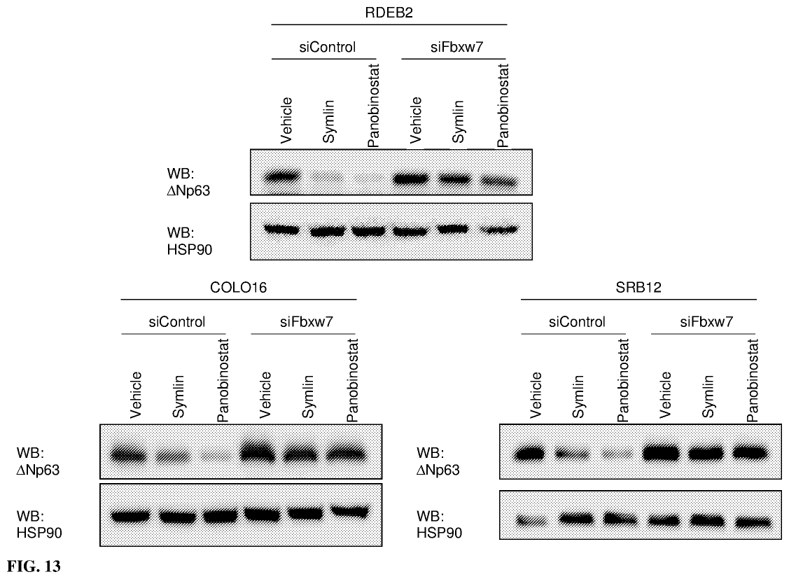

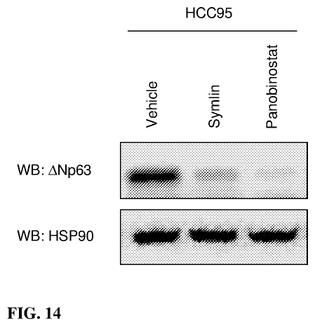

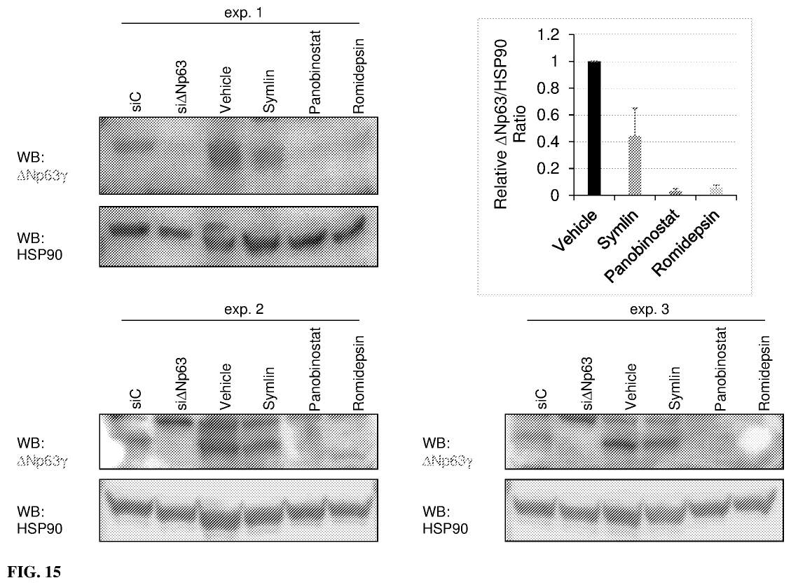

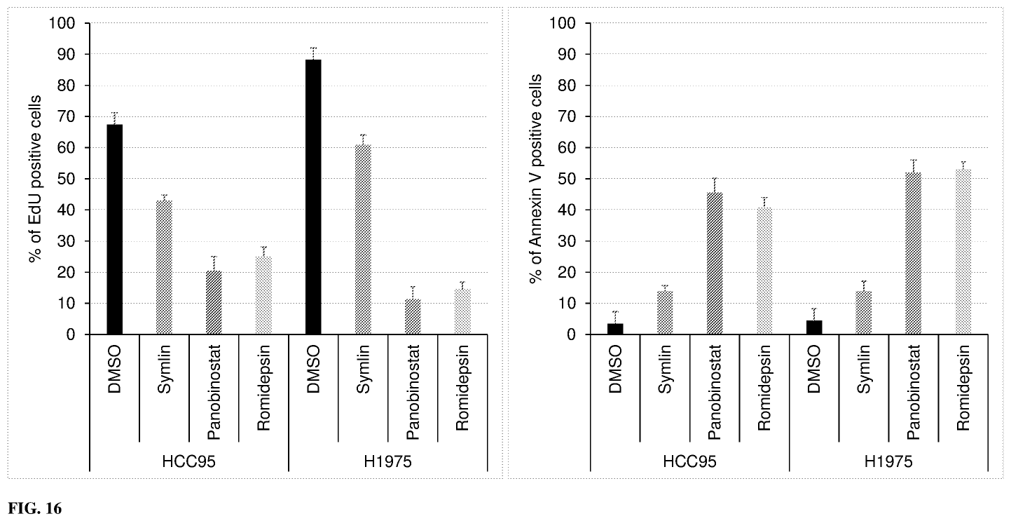

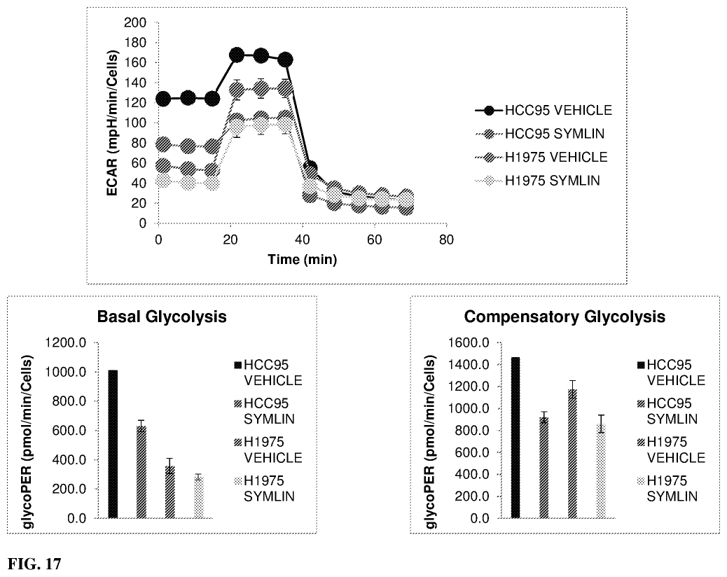

However, the ability to suppress ΔNp63 expression was affected by the expression of Fbxw7 which needs to be present for the suppressive effect of pramlintide to occur (FIG. 13). These same results were observed in the LUSC HCC95 lung cancer cell line (FIG. 14) showing the effect is not restricted to cuSCC. In fact, it was observed that both pramlintide and HDACi treatment reduces ΔNp63 in H1975 lung adenocarcinoma (LUAD) cells (FIG. 15). Investigating the effect that pramlintide of HDACi had on cell proliferation and survival of H1975 cells, it was observed that treatment with either pramlintide (symlin), panobinostat, or romidepsin, reduced proliferation in both HCC95 cells and H1975 cells. Additionally, all three treatments increased apoptosis as measured by annexin V staining (FIG. 16). To investigate what effects panobinostat has on the glycolytic rate of HCC95 and H1975 cells, glycolysis was measured by ECAR showing in both lung adenocarcinoma lines glycolysis was decreased with treatment (FIG. 17).

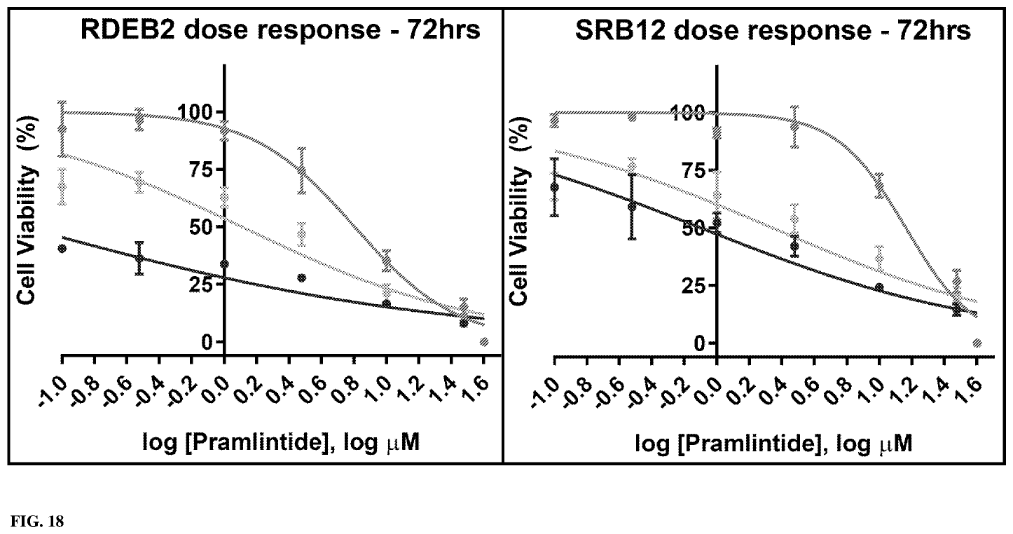

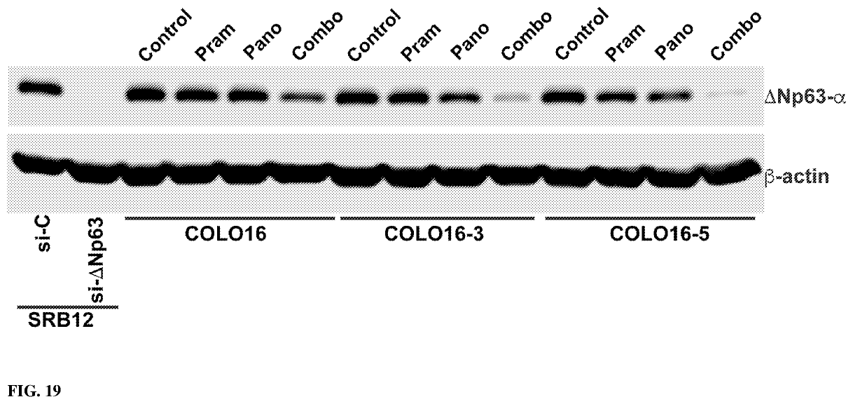

Wanting to see if other agents that were involved in metabolism had a similar effect on ΔNp63 expression or would have a synergistic effect when used in combination, the HDACi panobinostat was investigated for its effects on cancer. As shown in FIG. 18, while panobinostat did have an effect, the combination of pramlintide and panobinostat was far superior. As shown in FIG. 19, the combination of HDACi and pramlintide cooperatively suppresses ΔNp63 expression.

Accordingly, in one aspect, disclosed herein are methods of treating, inhibiting, reducing, and/or preventing a cancer or metastasis (such as for example, cutaneous squamous cell carcinoma (cuSCC) or lung cancer or any cancer comprising a p53, p63, or p73 mutation or aberrant expression thereof) in a subject comprising administering to the subject the combination therapy or the pharmaceutical composition any preceding aspect. For example, disclosed herein are methods of treating, inhibiting, reducing, and/or preventing a cancer or metastasis (such as for example, cutaneous squamous cell carcinoma (cuSCC) or lung cancer) in a subject comprising administering to the subject a first and second agent that inhibit ΔNp63 and/or ΔNp73; wherein the first agent comprises a glycolysis inhibitor (such as, for example, an amylin analog including but not limited to pramlintide); and wherein the second agent comprises an histone deacetylase (HDAC) inhibitor (HDACi) (such as, for example, hydroxamic acids including, but not limited to panobinostat, belinostat, trichostatin A, givinostat, resminostat, abexinostat, quisinostat, rocilinostat, practinostat, CHR-3996 and/or vorinostat; short chain fatty acids including, but not limited to valproic acid, butyric acid, and/or phenylbutyric acid; benzamides including, but not limited to entinostat, tacedinaline, 4SC202, and/or mocetinostat; cyclic tetrapeptides including, but not limited to romidepsin; and/or sirtuins inhibitors including, but not limited to nicotinamide, sirtinol, cambinol, and/or E-527). In one aspect, disclosed herein are methods of treating a cancer of any preceding aspect, wherein the method further comprises administering to the subject a radiation therapy.

In one aspect, disclosed herein are methods of increasing the sensitivity of a cancer (such as for example, cutaneous squamous cell carcinoma (cuSCC) or lung cancer or any cancer comprising a p53, p63, or p73 mutation or aberrant expression thereof) in a subject to histone deacetylase (HDAC) inhibitor (HDACi) therapy (such as, for example, hydroxamic acids including, but not limited to panobinostat, belinostat, trichostatin A, givinostat, resminostat, abexinostat, quisinostat, rocilinostat, practinostat, CHR-3996 and/or vorinostat; short chain fatty acids including, but not limited to valproic acid, butyric acid, and/or phenylbutyric acid; benzamides including, but not limited to entinostat, tacedinaline, 4SC202, and/or mocetinostat; cyclic tetrapeptides including, but not limited to romidepsin; and/or sirtuins inhibitors including, but not limited to nicotinamide, sirtinol, cambinol, and/or E-527) comprising administering to the subject an agent that inhibits glycolysis (such as, for example, an amylin analog including but not limited to pramlintide).

Also disclosed herein are methods of detecting sensitivity of a cancer (such as for example, cutaneous squamous cell carcinoma (cuSCC) or lung cancer or any cancer comprising a p53, p63, or p73 mutation or aberrant expression thereof) to a treatment that suppresses of ΔNp63 and/or ΔNp73 comprising detecting the presence of Fbxw7 expression; wherein the presence of Fbxw7 expression indicates sensitivity to a treatment that suppresses ΔNp63 and/or ΔNp73. In one aspect, the treatment that suppresses ΔNp63 and/or ΔNp73 comprises a glycolysis inhibitor (such as, for example, an amylin analog including but not limited to pramlintide) and/or a histone deacetylase (HDAC) inhibitor (HDACi) such as, for example, hydroxamic acids including, but not limited to panobinostat, belinostat, trichostatin A, givinostat, resminostat, abexinostat, quisinostat, rocilinostat, practinostat, CHR-3996 and/or vorinostat; short chain fatty acids including, but not limited to valproic acid, butyric acid, and/or phenylbutyric acid; benzamides including, but not limited to entinostat, tacedinaline, 4SC202, and/or mocetinostat; cyclic tetrapeptides including, but not limited to romidepsin; and/or sirtuins inhibitors including, but not limited to nicotinamide, sirtinol, cambinol, and/or E-527).

It is understood that as a decrease in CYR61 and CTGF and an increase of FXBO16 correlated with sensitivity to inhibitors of ΔNp63 and/or ΔNp73 and conversely an increase in CYR61 and CTGF and a decrease of FXBO16 correlated with resistance to inhibitors of ΔNp63 and/or ΔNp73 they can be used as biomarkers for sensitivity treatment with a glycolysis inhibitor and/or a HDACi. Thus, in one aspect, disclosed herein are methods of detecting sensitivity of a cancer (such as for example, cuSCC, HNSCC, or lung cancer) to a treatment that suppresses of ΔNp63 and/or ΔNp73 (such as, for example, an amylin analog including but not limited to pramlintide) and/or a histone deacetylase (HDAC) inhibitor (HDACi) such as, for example, hydroxamic acids including, but not limited to panobinostat, belinostat, trichostatin A, givinostat, resminostat, abexinostat, quisinostat, rocilinostat, practinostat, CHR-3996 and/or vorinostat; short chain fatty acids including, but not limited to valproic acid, butyric acid, and/or phenylbutyric acid; benzamides including, but not limited to entinostat, tacedinaline, 4SC202, and/or mocetinostat; cyclic tetrapeptides including, but not limited to romidepsin; and/or sirtuins inhibitors including, but not limited to nicotinamide, sirtinol, cambinol, and/or E-527) comprising obtaining a tissue sample, detecting the transcriptional level of connective tissue growth factor (CTGF), Cysteine-rich angiogenic inducer 61 (CYR61), or F-box protein 16 (FBXO16) in the tissue sample, wherein a decrease of CTFG or CYR61 or an increase of FBXO16 relative to a normal control indicates that the cancerous tissue is sensitive to a treatment that suppresses of ΔNp63; and wherein an increase of CTFG or CYR61 or a decrease of FBXO16 relative to a normal control indicates that the cancerous tissue is resistant to a treatment that suppresses of ΔNp63. Also disclosed herein are methods of detecting sensitivity of a cancer (such as for example, cuSCC, HNSCC, or lung cancer) to a treatment that suppresses of ΔNp63 and/or ΔNp73 (such as, for example, an amylin analog including but not limited to pramlintide) and/or a histone deacetylase (HDAC) inhibitor (HDACi) such as, for example, hydroxamic acids including, but not limited to panobinostat, belinostat, trichostatin A, givinostat, resminostat, abexinostat, quisinostat, rocilinostat, practinostat, CHR-3996 and/or vorinostat; short chain fatty acids including, but not limited to valproic acid, butyric acid, and/or phenylbutyric acid; benzamides including, but not limited to entinostat, tacedinaline, 4SC202, and/or mocetinostat; cyclic tetrapeptides including, but not limited to romidepsin; and/or sirtuins inhibitors including, but not limited to nicotinamide, sirtinol, cambinol, and/or E-527) comprising obtaining a tissue sample, contacting the tissue sample with a glycolysis inhibitor (such as, for example pramlintide); measuring the level of connective tissue growth factor (CTGF), Cysteine-rich angiogenic inducer 61 (CYR61), or F-box protein 16 (FBXO16) in the tissue sample, wherein a decrease of CTFG or CYR61 or an increase of FBXO16 relative to a normal control indicates that the cancerous tissue is sensitive to a treatment that suppresses of ΔNp63; and wherein no change or an increase of CTFG or CYR61 or a decrease of FBXO16 relative to a normal control indicates that the cancerous tissue is resistant to a treatment that suppresses of ΔNp63.

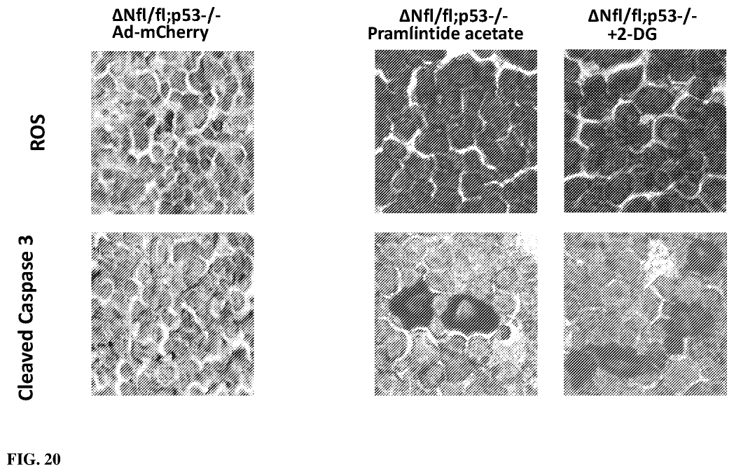

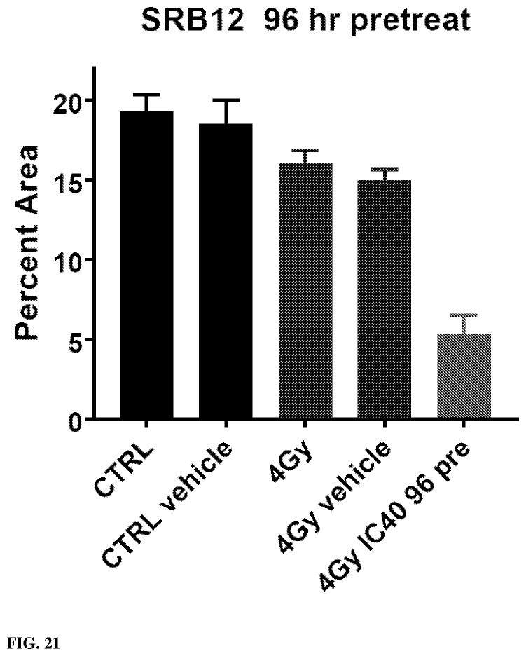

During the experiments it was observed that Pramlintide induces ROS in in-vivo in tumors (FIG. 20). To investigate whether because of its effect on ROS pramlintide would have a synergistic effect on radiation sensitivity, SRB12 cells were treated with radiation with and without the presence of pramlintide. These experiments showed that indeed cells were more sensitive to radiation treatment when also treated with pramlintide (FIG. 21). Accordingly, in one aspect, disclosed herein are methods of increasing the sensitivity of a cancer (such as for example, cutaneous squamous cell carcinoma (cuSCC) or lung cancer or any cancer comprising a p53, p63, or p73 mutation or aberrant expression thereof) in a subject to radiation therapy comprising administering to the subject an agent that inhibits ΔNp63 and/or ΔNp73. In one aspect the agent that inhibits ΔNp63 and/or ΔNp73 comprises an inhibitor of glycolysis (such as, for example, an amylin analog including but not limited to pramlintide).

The disclosed compositions can be used to treat, inhibit, reduce, and/or prevent any disease where uncontrolled cellular proliferation occurs such as cancers. A representative but non-limiting list of cancers that the disclosed compositions can be used to treat is the following: lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, lung cancers such as small cell lung cancer and non-small cell lung cancer, neuroblastoma/glioblastoma, ovarian cancer, skin cancer, liver cancer, melanoma, squamous cell carcinomas of the mouth, throat, larynx, and lung, cervical cancer, cervical carcinoma, breast cancer, and epithelial cancer, renal cancer, genitourinary cancer, pulmonary cancer, esophageal carcinoma, head and neck carcinoma, large bowel cancer, hematopoietic cancers; testicular cancer; colon cancer, rectal cancer, prostatic cancer, or pancreatic cancer.

Also disclosed herein are cancer treatment, inhibition, reduction, and/or prevention regimens comprising a) detecting the expression of Fxbw7 in the cancer (such as for example, cutaneous squamous cell carcinoma (cuSCC) or lung cancer or any cancer comprising a p53, p63, or p73 mutation or aberrant expression thereof); wherein expression of Fxbw7 indicates susceptibility of the cancer to inhibitors of ΔNp63 and/or ΔNp73; and b) when the presence of Fxbw7 expression is detected, the method further comprises administering to the subject a glycolysis inhibitor (such as, for example, an amylin analog including but not limited to pramlintide) and/or a histone deacetylase (HDAC) inhibitor (HDACi) (such as, for example, hydroxamic acids including, but not limited to panobinostat, belinostat, trichostatin A, givinostat, resminostat, abexinostat, quisinostat, rocilinostat, practinostat, CHR-3996 and/or vorinostat; short chain fatty acids including, but not limited to valproic acid, butyric acid, and/or phenylbutyric acid; benzamides including, but not limited to entinostat, tacedinaline, 4SC202, and/or mocetinostat; cyclic tetrapeptides including, but not limited to romidepsin; and/or sirtuins inhibitors including, but not limited to nicotinamide, sirtinol, cambinol, and/or E-527); or c) when the presence of Fxbw7 expression is not detected, the method further comprises administering to the subject a cancer regimen that does not include an agent that does not inhibit inhibitors of ΔNp63 and/or ΔNp73.

The disclosed treatment regimens when not utilizing inhibitors of ΔNp63 and/or ΔNp73 can include any anti-cancer therapy known in the art including, but not limited to Abemaciclib, Abiraterone Acetate, Abitrexate (Methotrexate), Abraxane (Paclitaxel Albumin-stabilized Nanoparticle Formulation), ABVD, ABVE, ABVE-PC, AC, AC-T, Adcetris (Brentuximab Vedotin), ADE, Ado-Trastuzumab Emtansine, Adriamycin (Doxorubicin Hydrochloride), Afatinib Dimaleate, Afinitor (Everolimus), Akynzeo (Netupitant and Palonosetron Hydrochloride), Aldara (Imiquimod), Aldesleukin, Alecensa (Alectinib), Alectinib, Alemtuzumab, Alimta (Pemetrexed Disodium), Aliqopa (Copanlisib Hydrochloride), Alkeran for Injection (Melphalan Hydrochloride), Alkeran Tablets (Melphalan), Aloxi (Palonosetron Hydrochloride), Alunbrig (Brigatinib), Ambochlorin (Chlorambucil), Amboclorin Chlorambucil), Amifostine, Aminolevulinic Acid, Anastrozole, Aprepitant, Aredia (Pamidronate Disodium), Arimidex (Anastrozole), Aromasin (Exemestane),Arranon (Nelarabine), Arsenic Trioxide, Arzerra (Ofatumumab), Asparaginase Erwinia chrysanthemi, Atezolizumab, Avastin (Bevacizumab), Avelumab, Axitinib, Azacitidine, Bavencio (Avelumab), BEACOPP, Becenum (Carmustine), Beleodaq (Belinostat), Belinostat, Bendamustine Hydrochloride, BEP, Besponsa (Inotuzumab Ozogamicin) , Bevacizumab, Bexarotene, Bexxar (Tositumomab and Iodine I 131 Tositumomab), Bicalutamide, BiCNU (Carmustine), Bleomycin, Blinatumomab, Blincyto (Blinatumomab), Bortezomib, Bosulif (Bosutinib), Bosutinib, Brentuximab Vedotin, Brigatinib, BuMel, Busulfan, Busulfex (Busulfan), Cabazitaxel, Cabometyx (Cabozantinib-S-Malate), Cabozantinib-S-Malate, CAF, Campath (Alemtuzumab), Camptosar , (Irinotecan Hydrochloride), Capecitabine, CAPDX, Carac (Fluorouracil--Topical), Carboplatin, CARBOPLATIN-TAXOL, Carfilzomib, Carmubris (Carmustine), Carmustine, Carmustine Implant, Casodex (Bicalutamide), CEM, Ceritinib, Cerubidine (Daunorubicin Hydrochloride), Cervarix (Recombinant HPV Bivalent Vaccine), Cetuximab, CEV, Chlorambucil, CHLORAMBUCIL-PREDNISONE, CHOP, Cisplatin, Cladribine, Clafen (Cyclophosphamide), Clofarabine, Clofarex (Clofarabine), Clolar (Clofarabine), CMF, Cobimetinib, Cometriq (Cabozantinib-S-Malate), Copanlisib Hydrochloride, COPDAC, COPP, COPP-ABV, Cosmegen (Dactinomycin), Cotellic (Cobimetinib), Crizotinib, CVP, Cyclophosphamide, Cyfos (Ifosfamide), Cyramza (Ramucirumab), Cytarabine, Cytarabine Liposome, Cytosar-U (Cytarabine), Cytoxan (Cyclophosphamide), Dabrafenib, Dacarbazine, Dacogen (Decitabine), Dactinomycin, Daratumumab, Darzalex (Daratumumab), Dasatinib, Daunorubicin Hydrochloride, Daunorubicin Hydrochloride and Cytarabine Liposome, Decitabine, Defibrotide Sodium, Defitelio (Defibrotide Sodium), Degarelix, Denileukin Diftitox, Denosumab, DepoCyt (Cytarabine Liposome), Dexamethasone, Dexrazoxane Hydrochloride, Dinutuximab, Docetaxel, Doxil (Doxorubicin Hydrochloride Liposome), Doxorubicin Hydrochloride, Doxorubicin Hydrochloride Liposome, Dox-SL (Doxorubicin Hydrochloride Liposome), DTIC-Dome (Dacarbazine), Durvalumab, Efudex (Fluorouracil--Topical), Elitek (Rasburicase), Ellence (Epirubicin Hydrochloride), Elotuzumab, Eloxatin (Oxaliplatin), Eltrombopag Olamine, Emend (Aprepitant), Empliciti (Elotuzumab), Enasidenib Mesylate, Enzalutamide, Epirubicin Hydrochloride, EPOCH, Erbitux (Cetuximab), Eribulin Mesylate, Erivedge (Vismodegib), Erlotinib Hydrochloride, Erwinaze (Asparaginase Erwinia chrysanthemi) , Ethyol (Amifostine), Etopophos (Etoposide Phosphate), Etoposide, Etoposide Phosphate, Evacet (Doxorubicin Hydrochloride Liposome), Everolimus, Evista , (Raloxifene Hydrochloride), Evomela (Melphalan Hydrochloride), Exemestane, 5-FU (Fluorouracil Injection), 5-FU (Fluorouracil-Topical), Fareston (Toremifene), Farydak (Panobinostat), Faslodex (Fulvestrant), FEC, Femara (Letrozole), Filgrastim, Fludara (Fludarabine Phosphate), Fludarabine Phosphate, Fluoroplex (Fluorouracil--Topical), Fluorouracil Injection, Fluorouracil--Topical, Flutamide, Folex (Methotrexate), Folex PFS (Methotrexate), FOLFIRI, FOLFIRI-BEVACIZUMAB, FOLFIRI-CETUXIMAB, FOLFIRINOX, FOLFOX, Folotyn (Pralatrexate), FU-LV, Fulvestrant, Gardasil (Recombinant HPV Quadrivalent Vaccine), Gardasil 9 (Recombinant HPV Nonavalent Vaccine), Gazyva (Obinutuzumab), Gefitinib, Gemcitabine Hydrochloride, GEMCITABINE-CISPLATIN, GEMCITABINE-OXALIPLATIN, Gemtuzumab Ozogamicin, Gemzar (Gemcitabine Hydrochloride), Gilotrif (Afatinib Dimaleate), Gleevec (Imatinib Mesylate), Gliadel (Carmustine Implant), Gliadel wafer (Carmustine Implant), Glucarpidase, Goserelin Acetate, Halaven (Eribulin Mesylate), Hemangeol (Propranolol Hydrochloride), Herceptin (Trastuzumab), HPV Bivalent Vaccine, Recombinant, HPV Nonavalent Vaccine, Recombinant, HPV Quadrivalent Vaccine, Recombinant, Hycamtin (Topotecan Hydrochloride), Hydrea (Hydroxyurea), Hydroxyurea, Hyper-CVAD, Ibrance (Palbociclib), Ibritumomab Tiuxetan, Ibrutinib, ICE, Iclusig (Ponatinib Hydrochloride), Idamycin (Idarubicin Hydrochloride), Idarubicin Hydrochloride, Idelalisib, Idhifa (Enasidenib Mesylate), Ifex (Ifosfamide), Ifosfamide, Ifosfamidum (Ifosfamide), IL-2 (Aldesleukin), Imatinib Mesylate, Imbruvica (Ibrutinib), Imfinzi (Durvalumab), Imiquimod, Imlygic (Talimogene Laherparepvec), Inlyta (Axitinib), Inotuzumab Ozogamicin, Interferon Alfa-2b, Recombinant, Interleukin-2 (Aldesleukin), Intron A (Recombinant Interferon Alfa-2b), Iodine I 131 Tositumomab and Tositumomab, Ipilimumab, Iressa (Gefitinib), Irinotecan Hydrochloride, Irinotecan Hydrochloride Liposome, Istodax (Romidepsin), Ixabepilone, Ixazomib Citrate, Ixempra (Ixabepilone), Jakafi (Ruxolitinib Phosphate), JEB, Jevtana (Cabazitaxel), Kadcyla (Ado-Trastuzumab Emtansine), Keoxifene (Raloxifene Hydrochloride), Kepivance (Palifermin), Keytruda (Pembrolizumab), Kisqali (Ribociclib), Kymriah (Tisagenlecleucel), Kyprolis (Carfilzomib), Lanreotide Acetate, Lapatinib Ditosylate, Lartruvo (Olaratumab), Lenalidomide, Lenvatinib Mesylate, Lenvima (Lenvatinib Mesylate), Letrozole, Leucovorin Calcium, Leukeran (Chlorambucil), Leuprolide Acetate, Leustatin (Cladribine), Levulan (Aminolevulinic Acid), Linfolizin (Chlorambucil), LipoDox (Doxorubicin Hydrochloride Liposome), Lomustine, Lonsurf (Trifluridine and Tipiracil Hydrochloride), Lupron (Leuprolide Acetate), Lupron Depot (Leuprolide Acetate), Lupron Depot-Ped (Leuprolide Acetate), Lynparza (Olaparib), Marqibo (Vincristine Sulfate Liposome), Matulane (Procarbazine Hydrochloride), Mechlorethamine Hydrochloride, Megestrol Acetate, Mekinist (Trametinib), Melphalan, Melphalan Hydrochloride, Mercaptopurine, Mesna, Mesnex (Mesna), Methazolastone (Temozolomide), Methotrexate, Methotrexate LPF (Methotrexate), Methylnaltrexone Bromide, Mexate (Methotrexate), Mexate-AQ (Methotrexate), Midostaurin, Mitomycin C, Mitoxantrone Hydrochloride, Mitozytrex (Mitomycin C), MOPP, Mozobil (Plerixafor), Mustargen (Mechlorethamine Hydrochloride) , Mutamycin (Mitomycin C), Myleran (Busulfan), Mylosar (Azacitidine), Mylotarg (Gemtuzumab Ozogamicin), Nanoparticle Paclitaxel (Paclitaxel Albumin-stabilized Nanoparticle Formulation), Navelbine (Vinorelbine Tartrate), Necitumumab, Nelarabine, Neosar (Cyclophosphamide), Neratinib Maleate, Nerlynx (Neratinib Maleate), Netupitant and Palonosetron Hydrochloride, Neulasta (Pegfilgrastim), Neupogen (Filgrastim), Nexavar (Sorafenib Tosylate), Nilandron (Nilutamide), Nilotinib, Nilutamide, Ninlaro (Ixazomib Citrate), Niraparib Tosylate Monohydrate, Nivolumab, Nolvadex (Tamoxifen Citrate), Nplate (Romiplostim), Obinutuzumab, Odomzo (Sonidegib), OEPA, Ofatumumab, OFF, Olaparib, Olaratumab, Omacetaxine Mepesuccinate, Oncaspar (Pegaspargase), Ondansetron Hydrochloride, Onivyde (Irinotecan Hydrochloride Liposome), Ontak (Denileukin Diftitox), Opdivo (Nivolumab), OPPA, Osimertinib, Oxaliplatin, Paclitaxel, Paclitaxel Albumin-stabilized Nanoparticle Formulation, PAD, Palbociclib, Palifermin, Palonosetron Hydrochloride, Palonosetron Hydrochloride and Netupitant, Pamidronate Disodium, Panitumumab, Panobinostat, Paraplat (Carboplatin), Paraplatin (Carboplatin), Pazopanib Hydrochloride, PCV, PEB, Pegaspargase, Pegfilgrastim, Peginterferon Alfa-2b, PEG-Intron (Peginterferon Alfa-2b), Pembrolizumab, Pemetrexed Disodium, Perjeta (Pertuzumab), Pertuzumab, Platinol (Cisplatin), Platinol-AQ (Cisplatin), Plerixafor, Pomalidomide, Pomalyst (Pomalidomide), Ponatinib Hydrochloride, Portrazza (Necitumumab), Pralatrexate, Prednisone, Procarbazine Hydrochloride, Proleukin (Aldesleukin), Prolia (Denosumab), Promacta (Eltrombopag Olamine), Propranolol Hydrochloride, Provenge (Sipuleucel-T), Purinethol (Mercaptopurine), Purixan (Mercaptopurine), Radium 223 Dichloride, Raloxifene Hydrochloride, Ramucirumab, Rasburicase, R-CHOP, R-CVP, Recombinant Human Papillomavirus (HPV) Bivalent Vaccine, Recombinant Human Papillomavirus (HPV) Nonavalent Vaccine, Recombinant Human Papillomavirus (HPV) Quadrivalent Vaccine, Recombinant Interferon Alfa-2b, Regorafenib, Relistor (Methylnaltrexone Bromide), R-EPOCH, Revlimid (Lenalidomide), Rheumatrex (Methotrexate), Ribociclib, R-ICE, Rituxan (Rituximab), Rituxan Hycela (Rituximab and Hyaluronidase Human), Rituximab, Rituximab and , Hyaluronidase Human, Rolapitant Hydrochloride, Romidepsin, Romiplostim, Rubidomycin (Daunorubicin Hydrochloride), Rubraca (Rucaparib Camsylate), Rucaparib Camsylate, Ruxolitinib Phosphate, Rydapt (Midostaurin), Sclerosol Intrapleural Aerosol (Talc), Siltuximab, Sipuleucel-T, Somatuline Depot (Lanreotide Acetate), Sonidegib, Sorafenib Tosylate, Sprycel (Dasatinib), STANFORD V, Sterile Talc Powder (Talc), Steritalc (Talc), Stivarga (Regorafenib), Sunitinib Malate, Sutent (Sunitinib Malate), Sylatron (Peginterferon Alfa-2b), Sylvant (Siltuximab), Synribo (Omacetaxine Mepesuccinate), Tabloid (Thioguanine), TAC, Tafinlar (Dabrafenib), Tagrisso (Osimertinib), Talc, Talimogene Laherparepvec, Tamoxifen Citrate, Tarabine PFS (Cytarabine), Tarceva (Erlotinib Hydrochloride), Targretin (Bexarotene), Tasigna (Nilotinib), Taxol (Paclitaxel), Taxotere (Docetaxel), Tecentriq , (Atezolizumab), Temodar (Temozolomide), Temozolomide, Temsirolimus, Thalidomide, Thalomid (Thalidomide), Thioguanine, Thiotepa, Tisagenlecleucel, Tolak (Fluorouracil--Topical), Topotecan Hydrochloride, Toremifene, Torisel (Temsirolimus), Tositumomab and Iodine I 131 Tositumomab, Totect (Dexrazoxane Hydrochloride), TPF, Trabectedin, Trametinib, Trastuzumab, Treanda (Bendamustine Hydrochloride), Trifluridine and Tipiracil Hydrochloride, Trisenox (Arsenic Trioxide), Tykerb (Lapatinib Ditosylate), Unituxin (Dinutuximab), Uridine Triacetate, VAC, Vandetanib, VAMP, Varubi (Rolapitant Hydrochloride), Vectibix (Panitumumab), VeIP, Velban (Vinblastine Sulfate), Velcade (Bortezomib), Velsar (Vinblastine Sulfate), Vemurafenib, Venclexta (Venetoclax), Venetoclax, Verzenio (Abemaciclib), Viadur (Leuprolide Acetate), Vidaza (Azacitidine), Vinblastine Sulfate, Vincasar PFS (Vincristine Sulfate), Vincristine Sulfate, Vincristine Sulfate Liposome, Vinorelbine Tartrate, VIP, Vismodegib, Vistogard (Uridine Triacetate), Voraxaze (Glucarpidase), Vorinostat, Votrient (Pazopanib Hydrochloride), Vyxeos (Daunorubicin Hydrochloride and Cytarabine Liposome), Wellcovorin (Leucovorin Calcium), Xalkori (Crizotinib), Xeloda (Capecitabine), XELIRI, XELOX, Xgeva (Denosumab), Xofigo (Radium 223 Dichloride), Xtandi (Enzalutamide), Yervoy (Ipilimumab), Yondelis (Trabectedin), Zaltrap (Ziv-Aflibercept), Zarxio (Filgrastim), Zejula (Niraparib Tosylate Monohydrate), Zelboraf (Vemurafenib), Zevalin (Ibritumomab Tiuxetan), Zinecard (Dexrazoxane Hydrochloride), Ziv-Aflibercept, Zofran (Ondansetron Hydrochloride), Zoladex (Goserelin Acetate), Zoledronic Acid, Zolinza (Vorinostat), Zometa (Zoledronic Acid), Zydelig (Idelalisib), Zykadia (Ceritinib), and/or Zytiga (Abiraterone Acetate). Where an EGFR splice variant isoform is not detected, the treatment methods can include or further include checkpoint inhibitors include, but are not limited to antibodies that block PD-1 (Nivolumab (BMS-936558 or MDX1106), CT-011, MK-3475), PD-L1 (MDX-1105 (BMS-936559), MPDL3280A, or MSB0010718C), PD-L2 (rHIgM12B7), CTLA-4 (Ipilimumab (MDX-010), Tremelimumab (CP-675,206)), IDO, B7-H3 (MGA271), B7-H4, TIM3, LAG-3 (BMS-986016). Where the presence of an EGFR splice variant isoform is detected the treatment regimen implemented does not include a immune checkpoint blockade inhibitor. It is understood and herein recognized that the presence of an EGFR splice variant isoform does not necessarily indicate that the cancer is resistant to all immune checkpoint blockade inhibitors. In one aspect, the detection of the EGFR splice variant isoform indicates resistance to PD-1, PD-L1, PD-12, CRLA-4, IDO, B7-H3, B7-H4, TIM3, or LAG-3. In one aspect, the detection of the EGFR splice variant isoform indicates resistance to PD-L1. Thus, when resistance is only to a particular form of immune checkpoint blockade inhibition (such as, for example PD-L1), other immune checkpoint blockade inhibitors can still be used.

C. Compositions

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular glycolysis inhibitor or HDAC inhibitor is disclosed and discussed and a number of modifications that can be made to a number of molecules including the glycolysis inhibitor or HDAC inhibitor are discussed, specifically contemplated is each and every combination and permutation of glycolysis inhibitor or HDAC inhibitor and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

In one aspect disclosed herein are combination therapies comprising a glycolysis inhibitor (such as, for example, an amylin analog including but not limited to pramlintide) and an histone deacetylase (HDAC) inhibitor (HDACi) (such as, for example, hydroxamic acids including, but not limited to panobinostat, belinostat, trichostatin A, givinostat, resminostat, abexinostat, quisinostat, rocilinostat, practinostat, CHR-3996 and/or vorinostat; short chain fatty acids including, but not limited to valproic acid, butyric acid, and/or phenylbutyric acid; benzamides including, but not limited to entinostat, tacedinaline, 4SC202, and/or mocetinostat; cyclic tetrapeptides including, but not limited to romidepsin; and/or sirtuins inhibitors including, but not limited to nicotinamide, sirtinol, cambinol, and/or E-527). It is understood and herein contemplated that the glycolysis inhibitor and the HDACi can be administered sequentially in any order, concurrently as separate compositions, or simultaneously as components of a single composition, such as a pharmaceutical composition. 1. Pharmaceutical carriers/Delivery of pharmaceutical products

As described above, the compositions can also be administered in vivo in a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art. Accordingly, disclosed herein are pharmaceutical compositions comprising a glycolysis inhibitor (such as, for example, an amylin analog including but not limited to pramlintide) and an histone deacetylase (HDAC) inhibitor (HDACi) (such as, for example, hydroxamic acids including, but not limited to panobinostat, belinostat, trichostatin A, givinostat, resminostat, abexinostat, quisinostat, rocilinostat, practinostat, CHR-3996 and/or vorinostat; short chain fatty acids including, but not limited to valproic acid, butyric acid, and/or phenylbutyric acid; benzamides including, but not limited to entinostat, tacedinaline, 4SC202, and/or mocetinostat; cyclic tetrapeptides including, but not limited to romidepsin; and/or sirtuins inhibitors including, but not limited to nicotinamide, sirtinol, cambinol, and/or E-527).

The compositions may be administered orally, parenterally (e.g., intravenously), by intramuscular injection, by intraperitoneal injection, transdermally, extracorporeally, topically or the like, including topical intranasal administration or administration by inhalant. As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

a) Pharmaceutically Acceptable Carriers

The compositions, including antibodies, can be used therapeutically in combination with a pharmaceutically acceptable carrier.

Suitable carriers and their formulations are described in Remington: The Science and Practice of Pharmacy (19th ed.) ed. A. R. Gennaro, Mack Publishing Company, Easton, Pa. 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, antiinflammatory agents, anesthetics, and the like.

The pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid- or base- addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines b) Therapeutic Uses

Effective dosages and schedules for administering the compositions may be determined empirically, and making such determinations is within the skill in the art. The dosage ranges for the administration of the compositions are those large enough to produce the desired effect in which the symptoms of the disorder are effected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products. For example, guidance in selecting appropriate doses for antibodies can be found in the literature on therapeutic uses of antibodies, e.g., Handbook of Monoclonal Antibodies, Ferrone et al., eds., Noges Publications, Park Ridge, N.J., (1985) ch. 22 and pp. 303-357; Smith et al., Antibodies in Human Diagnosis and Therapy, Haber et al., eds., Raven Press, New York (1977) pp. 365-389. A typical daily dosage of the antibody used alone might range from about 1 ug/kg to up to 100 mg/kg of body weight or more per day, depending on the factors mentioned above.

### D. REFERENCES

Venkatanarayan et al. Nature 2015; 517:626-30

1. A combination therapy comprising a glycolysis inhibitor and a histone deacetylase (HDAC) inhibitor (HDACi).

2. The combination therapy of claim 1, wherein the glycolysis inhibitor comprises pramlintide.

3. The combination therapy of claim 1, wherein the HDACi comprises panobinostat.

4. A pharmaceutical composition comprising a glycolysis inhibitor and a histone deacetylase (HDAC) inhibitor (HDACi) the combination therapy of claim 1.

5. A method of treating a cancer in a subject comprising administering to the subject the combination therapy of claim 1.

6. The method of claim 5, wherein the cancer comprises cutaneous squamous cell carcinoma, head and neck squamous cell carcinoma, or lung cancer.

7. (canceled)

8. (canceled)

9. (canceled)

10. (canceled)

11. The method of claim 6, wherein the method further comprises administering to the subject a radiation therapy.

12. A cancer treatment regimen comprising
a. detecting the expression of Fxbw7 in a cancer; wherein expression of Fxbw7 indicates susceptibility of the cancer to inhibitors of ΔNp63 and/or ΔNp73.
b. when the presence of Fxbw7 expression is detected, the method further comprises administering to the subject a glycolysis inhibitor and/or a histone deacetylase (HDAC) inhibitor (HDACi);
c. when the presence of Fxbw7 expression is not detected, the method further comprises administering to the subject a cancer regimen that does not include an agent that does not inhibit inhibitors of ΔNp63 and/or ΔNp73.

13. A method of increasing the sensitivity of a cancer in a subject to histone deacetylase (HDAC) inhibitor (HDACi) therapy comprising administering to the subject an agent that inhibits glycolysis.

14. The method of claim 13, wherein the glycolysis inhibitor comprises pramlintide.

15. The method of claim 13, wherein administration of the glycolysis inhibitor decrease the inhibitory concentration of the HDACi needed to be effective against the cancer.

16. A method of increasing the sensitivity of a cancer in a subject to radiation therapy comprising administering to the subject an agent that inhibits ΔNp63 and/or ΔNp73.

17. The method of claim 16, wherein the agent comprises pramlintide.

18. A method of detecting sensitivity of a cancer to a treatment that suppresses of ΔNp63 and/or ΔNp73 comprising obtaining a cancerous tissue sample, detecting the presence of Fbxw7 expression in the tissue sample and/or detecting the transcriptional level of connective tissue growth factor (CTGF), Cysteine-rich angiogenic inducer 61 (CYR61), or F-box protein 16 (FBXO16) in the tissue sample; wherein the presence of Fbxw7 expression indicates sensitivity to a treatment that suppresses ΔNp63 and/or ΔNp73; wherein a decrease of CTFG or CYR61 or an increase of FBXO16 relative to a normal control indicates that the cancerous tissue is sensitive to a treatment that suppresses of ΔNp63; and wherein an increase of CTFG or CYR61 or a decrease of FBXO16 relative to a normal control indicates that the cancerous tissue is resistant to a treatment that suppresses of ΔNp63.

19. The method of claim 17, wherein the treatment that suppresses ΔNp63 and/or ΔNp73 comprises a glycolysis inhibitor and/or a histone deacetylase (HDAC) inhibitor (HDACi).

20. The method of claim 18, wherein the glycolysis inhibitor comprises pramlintide.

21. The method of claim 18, wherein the HDACi comprises panobinostat.

22. The method of claim 17, wherein the cancer comprises cutaneous squamous cell carcinoma, head and neck squamous cell carcinoma, or lung cancer.

23. (canceled)

24. (canceled)

25. A method of detecting sensitivity of a cancer to a treatment that suppresses of ΔNp63 and/or ΔNp73 comprising obtaining a tissue sample, contacting the tissue sample with a glycolysis inhibitor; measuring the level of connective tissue growth factor (CTGF), Cysteine-rich angiogenic inducer 61 (CYR61), or F-box protein 16 (FBXO16) in the tissue sample, wherein a decrease of CTFG or CYR61 or an increase of FBXO16 relative to a normal control indicates that the cancerous tissue is sensitive to a treatment that suppresses of ΔNp63; and
wherein no change or an increase of CTFG or CYR61 or a decrease of FBXO16 relative to a normal control indicates that the cancerous tissue is resistant to a treatment that suppresses of ΔNp63.

26. The method of claim 25, wherein the glycolysis inhibitor is pramlintide.
